(19) United States
(12) Patent Application Publication
(10) Pub. No.: US 2023/0187084 A1
(43) Pub. Date: Jun. 15, 2023

(54) DRUG INFORMATION AGGREGATION

(71) Applicant: Raghu Eachambadi DESIKAN, Manhasset, NY (US)

(72) Inventor: Raghu Eachambadi DESIKAN, Manhasset, NY (US)

(21) Appl. No.: 18/065,092

(22) Filed: Dec. 13, 2022

Related U.S. Application Data
(60) Provisional application No. 63/265,390, filed on Dec. 14, 2021.

(51) Int. Cl. G16H 70/40

(52) U.S. Cl. CPC G16H 70/40

(57) ABSTRACT

The present subject matter is for the identification of interaction between drugs and providing drug side effect education and information. Provided is a drug information aggregation system (102) that comprises a communication module (104) that accesses health data of patients based on healthcare plans of the patients. The communication module (104) further interacts with a plurality of publicly and privately accessible information sources to retrieve various information relating to the one or more drugs. The system (102) further comprises an information aggregation module (108) that creates and stores a drug information database (110). Furthermore, a side effect information module (112) provides side effects information based on the drug information database (110).

### PRIORITY CLAIM

This application claims the benefit of priority to U.S. Provisional Patent Application Ser. No. 63/265,390, filed Dec. 14, 2021, and entitled “DRUG INFORMATION AGGREGATION SYSTEM,” which provisional application is incorporated herein by reference in its entirety.

### TECHNICAL FIELD

The present subject matter relates generally to information aggregation and more particularly to aggregation of information relating to drugs for identification of interaction between drugs and to provide drug side effect education and information.

### BACKGROUND

There has been a steady increase in the number of people with chronic diseases which frequently involve multiple long-term drugs/polypharmacy, and/or complex therapies and regimens. Inadequate disease management may lead to disease progression, health complications, financial, and societal consequences.

Poor adherence to drugs is often a primary cause of inadequate disease management. Many chronically treated patients do not adhere to their prescription drugs, and many lack an understanding of the importance of adherence and self-care. Factors for poor adherence may include the perceived inconvenience of dosing regimen; forgetfulness; potential side effects from drugs, drug-drug interactions, and other supplements; in addition to factors, such as the ongoing toll on poor health; and not feeling improvement of symptoms despite improvements in clinical measurements, among others.

### BRIEF DESCRIPTION OF THE DRAWINGS

The following detailed description references the drawings, wherein:

FIG. 1 illustrates a network environment comprising a drug information aggregation system, in accordance with an example implementation of the present subject matter;

FIG. 2 illustrates a communication module of the drug information aggregation system in communication with information sources, in accordance with an example implementation of the present subject matter;

FIG. 3 illustrates the drug information aggregation system, in accordance with an example implementation of the present subject matter;

FIG. 4 illustrates a method for information aggregation within a networked environment, in accordance with an example implementation of the present subject matter; and

FIG. 5 illustrates a computing environment for information aggregation, according to an example implementation of the present subject matter.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

Many people with ongoing, chronic medical conditions are often prescribed drugs to take on a regular basis to manage their health. Each condition may require one or more drugs to specifically manage that condition, and when that alone is inadequate to control the disease, the drug may be replaced, have dosage increased, or an additional drug may be added.

Side effects with varying degrees of severity are likely to happen when a patient uses a drug, or when the drug dosage is changed. Each drug has its own unique side effect profile that may be provided on the drug's drug label, and the onset of these side effects may vary. There are different causes for side effects linked to drugs which may include dosage, which may need adjusting; an individual's reaction to an ingredient in the drug; etc. Side effects, if not managed properly, can be a reason why a patient may stop taking his/her drugs, which can lead to deterioration in his/her health condition.

In addition to the prescribed drugs, patients may consume a variety of supplements as well as over-the-counter drugs for minor ailments that the patients may think do not warrant a visit to a medical professional and hence may self-medicate. The patients may consume other non-prescription agents for general wellness, ranging from, for example, vitamins to herbs.

Patients consuming several drugs may be prone to atypical clinical events, examples of which include drug-drug interactions (e.g., one drug altering the pharmacokinetics of another drug), drug-food interactions, drug-gene interactions, drug-supplement interactions, and drug-allergy reactions.

Traditionally, medical professionals are expected to have knowledge relating to side effects including potential adverse drug reactions, pharmacology, and pharmacogenetics, or to have access to such information from published sources, such as textbooks, reference manuals, etc., that are usually not accessible from a single source and are increasingly complex. However, it is no longer sufficient to merely publish information in the form of a textbook or a reference manual, or an isolated online journal.

Even informed patients who are on multiple drugs regimen may face difficulties in remembering to take the drug as per recommended drug regimen. Sometimes, patients may have difficulty remembering that they have already taken a dose. Some patients have difficulty remembering the recommended dose of a drug to be taken. Such situations may lead to poor adherence or poor compliance and potentially can lead to non-compliance, or non-adherence to the prescribed drug regimen by the patients. A general lack of compliance to a prescribed drug regimen, coupled with a lack of general knowledge amongst the patients on how the body absorbs and uses drugs and hence ignoring potential side effects and confusing the side effects as general malaise, may result in patients taking multiple drugs in a manner that may cause potential adverse drug interactions. Some drug interactions may occur causing the patients to experience unexpected side effects. The drug interactions may also result in reduced efficacy or increased side effects for one or more of the drugs.

In such adverse situations, it may not always be possible for patients or their respective caregivers to reach out to the medical professional or search on the internet to gain knowledge relating to side effects or other potential adverse drug reactions, pharmacology, and pharmacogenetics, or to have access to such information from various knowledge resources that are not accessible from a single source and are usually complex to comprehend.

An example of a possible unintended drug interaction error with unintended health consequences may be when a patient is taking a calcium channel blocker for hypertension but goes out to another physician (e.g., due to a health issue while on vacation), who does not have access to the patient's prior medical records, with sharp muscle spasms in a lower back and not being able to recall the list of medications that the patient has been consuming for treating hypertension. In such a case, if the patient were to be prescribed a corticosteroid for relief of the muscle spasms, the corticosteroid drug has the potential to interfere with the calcium channel blocker to cause immunosuppressive reactions which may cause further unintended health consequences, thus causing an occurrence of drug interaction error.

Another example of drug interaction error may be when the same hypertensive patient, facing some sudden pain, decides to self-medicate with an over-the-counter pain drug in the non-steroidal anti-inflammatory class (NSAIDs) category, e.g., naproxen, ibuprofen, diclofenac, etc., while being on medication for hypertension. In this instance, the patient may unknowingly increase his or her risk of gastrointestinal or renal toxic reactions.

Further still, the current pre-packaging of many drugs does not provide a simplified labeling technique that may permit the patients or their caregivers to understand a drug's potential side effects or interactions with other substances. Additionally, although a pharmacist may provide information about drug interactions or side effects, no simple, clear, and easily accessible record exists to describe such information in a patient-friendly manner. Moreover, there is a risk of patients receiving unverified or false/inaccurate information from the internet that may be contrary to generally established and accepted medical advice/recommendations.

While some standalone drug education and adherence programs are available for a few branded drugs from their manufacturers, they are specific to only that drug. Support programs that provide drug education may not generally be available for generic drugs which account for nearly 9 out of 10 prescriptions today. Furthermore, support programs to provide drug and disease education, i.e., aggregation of information relating to the diseases patient(s) have, the multiple drugs and supplements that a user may be consuming, for side effect education, and information and/or identification of the interaction between drugs in a wholistic manner, do not presently exist.

Thus, there exists no single source of information from where the patients may get customized education including information on their condition(s)/disease(s), prescription drugs, advice, and support relating to expected side effects due to the reaction of the body to the drug in simple, easy-to-understand language to help them in managing their specific medical needs.

There is thus a need for a system that is designed to store and process metabolic, pharmacologic, and pharmacogenetic data of various drugs at the same location; interpret that data in the context of patient-specific clinical factors; make that data readily available and distribute it to the patients or their authorized caregivers in a user-friendly and easy to understand manner.

Example approaches for providing information relating to drug interactions and side effects in an efficient manner, avoiding the need to procure such information from a variety of disparate sources, are described. In an example, a drug information aggregation system is provided that facilitates drug information aggregation, that is, enables drug interaction information and side effects information to be accessed. The side effect may be understood as an undesirable secondary effect that may occur in addition to the desired therapeutic effect of a drug. The drug interaction, on the other hand, may be understood as a change in the action of a drug caused by concomitant administration with another drug or supplement, including food or beverage; and may be one of the reasons for side effects linked to drugs.

In an example implementation, the drug information aggregation system comprises a processor and a memory in communication with the processor. The system further comprises a communication module, coupled to the processor, that is configured to access the health data of patients based on healthcare plans of the patients. The health data of a patient includes clinical demographic data of each patient and their relevant health conditions, such as medical records of the patient comprising information relating to drugs that are prescribed to the patient by medical professionals. The patients may be enrolled in one or more healthcare plans provided through a combination of private health insurance and/or public health coverage. In some cases, patients may have two healthcare plan providers, one being theirs and the other being their spouses as a secondary healthcare plan. In an example implementation of the present subject matter, the health data of the patients may be retrieved from one or more servers and/or databases maintained by one or more healthcare plan providers of the patients.

The communication module is further configured to interact with a plurality of publicly and privately accessible information sources to retrieve and aggregate information relating to side effects and/or drug interactions that may potentially occur when a drug prescribed to a patient is consumed either alone or with other drugs, or supplements.

In accordance with an example implementation of the present subject matter, the communication module is configured to retrieve drug approval information relating to the drugs from prescribing information (PI) documents, wherein the PI documents include information provided by drug regulatory authorities relating to the drugs and their uses. In an example, the communication module also retrieves educational information relating to one or more drugs from a plurality of preidentified web sources. The educational information includes publications relating to the prescribed drugs and medical conditions that are associated with the drugs.

The communication module may also retrieve drug history information relating to the prescribed drugs, in one implementation. The drug history information of a prescribed drug may comprise information indicative of the history of development of the drug. The drug history information includes various information, such as drug class, name of the drug, date of approval by the drug regulatory authorities, date when the drug was first made available for public use, name of manufacturers, original manufacturer, pricing, and a number of clinical publications.

Furthermore, in an example, the communication module may additionally retrieve mechanism-of-action information relating to the drugs. The mechanism-of-action information of a drug includes the theoretical or proposed mechanism-of-action of molecules present in the drug.

In another example, the communication module may optionally retrieve emerging trends information. The emerging trends information may be understood as data comprising information relating to the medical conditions associated with one or more drugs prescribed to the patient. The emerging trends information may include national and international clinical guidelines for managing medical conditions, in an example.

The information retrieved by the communication module from one or more of the various above-described sources is aggregated in a manner that information relating to drug interactions may be easily retrievable by a user querying the drug information aggregation system for such information. For this purpose, the drug information aggregation system is further provided with an expert input module. The expert input module allows experts, such as medical professionals and researchers to interact with the drug information aggregation system to process the information retrieved by the communication module. Accordingly, in an example, the expert input module receives expert inputs relating to the information retrieved by the communication module.

An information aggregation module of the drug information aggregation system constructs and stores, in a memory, a drug information database. The drug information database is created based on the inputs which are provided by the experts by processing the data that is collected by the communication module from various publicly and privately accessible information sources.

The drug information aggregation system provides users, who may be patients, or their caregivers, with information pertaining to the side effects of a drug prescribed to the patient, or of any additional drug or supplement that may include OTC drugs or dietary supplements. A side effect information module of the drug information aggregation system provides side effects information based on the data collected in the drug information database. The side effects information is indicative of adverse events, unwanted or unexpected events, or reactions that may occur upon consumption of a prescribed drug, additional drug, or supplement.

The users are provided with relevant information pertaining to drug interactions when they query the drug information aggregation system to seek information pertaining to the interaction of the drug prescribed to them with at least one additional drug or supplement. An interaction information module of the drug information aggregation system provides drug interaction information based on the data collected in the drug information database. The drug interaction information is indicative of a change in pharmacokinetic parameters of a drug when the drug is consumed with the at least one additional drug or supplement. Given that each drug may have its known and unique side effect profile, patients are informed of potential side effects along with advice to contact their healthcare provider if these side effects do not go away or are difficult to tolerate.

Thus, the drug information aggregation system enables the detection of potential adverse drug reactions that may occur in patients who are on multiple drugs regimen and, in an example, help them with a description of a recommended alternate course of action according to expected side effects or detected interaction. For instance, in case of a potential adverse reaction to an OTC drug, the patient may be offered alternative suggestions while also recommending that the patient confirm the appropriateness with their healthcare provider, if applicable.

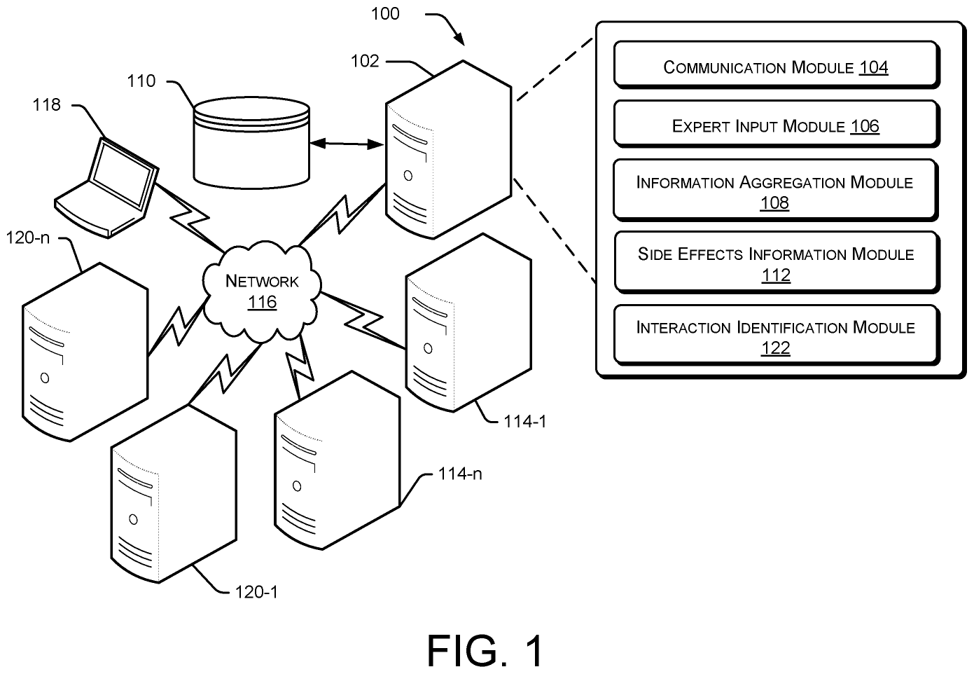

The above-described drug information aggregation system is further described with reference to FIGS. 1 to 5. It should be noted that the description and figures merely illustrate the principles of the present subject matter along with examples described herein and should not be construed as a limitation to the present subject matter. It is thus noted that various arrangements may be devised that, although not explicitly described or shown herein, describe the principles of the present subject matter. Moreover, all statements herein reciting principles, aspects, and examples of the present subject matter, as well as specific examples thereof, are intended to encompass equivalents thereof.

FIG. 1 illustrates a network environment 100 comprising drug information aggregation system 102, according to an example of the present subject matter. The drug information aggregation system 102 disseminates information to patients about their medical condition as well the therapeutic and dosing details of the prescribed drug(s) with an emphasis on side effects and interaction information relating to potential drug interactions between the prescribed drug(s) and commonly consumed food, supplements as well as additional drug(s). Thus, the drug information aggregation system 102 provides for increasing patients' understanding of their ongoing medical treatment, drug safety awareness, continued drug adherence, and avoiding adverse events, such as drug interactions and other side effect events driven by interruptions or discontinuation of prescribed medical treatment.

In an example, prescribed drugs, which include both generic drugs and brand-name drugs, may be understood as drugs that may usually be obtained by means of a medical professional's prescription. Examples of prescription drugs may include but are not limited to Metformin, Levothyroxine, Lisinopril, Vicodin, Synthroid, Levoxyl, Lipitor, Norvasc, Simvastatin, Glyburide, etc.

In an example, an additional drug, also known as OTC (over the counter) drug, may be understood as any drug that may be bought without a medical professional's prescription. Examples of the additional drug may include but are not limited to pain relievers, such as acetaminophen and ibuprofen, cough suppressants, such as dextromethorphan, and antihistamines like loratadine.

Similarly, in an example, supplements may be considered products that are intended to supplement one's diet. As indicated above, examples of supplements may include herbs, vitamins, minerals, proteins, amino acids, bodybuilding supplements, essential fatty acids, natural products, and probiotics that may be available in the form of pills, capsules, tablets, powder, or liquid, etc.

The term “side effects information”, as used in the present description, comprises information pertaining to any undesirable secondary effects of at least one of one or more drugs prescribed to a patient, at least one additional drug or supplement, that may occur in addition to a desired therapeutic effect of the prescribed drug, additional drug or supplement.

The term “drug interaction information”, as used in the present description comprises information pertaining to a decrease or increase in the effectiveness of a drug, augmented side effects of the drug, or change in the action or side effects of the drug caused by concomitant administration of the drug with additional drug or supplement(s). In an example, the drug interaction information comprises information relating to drug-drug interactions, drug-food interactions, and drug-disease interactions.

Drug interaction is one of the side effects that may occur, for example, when two or more drugs react with each other. Such drugs may be from a combination of the drugs prescribed to the patient and/or OTC drugs. For instance, consuming digoxin with antibiotics like erythromycin or clarithromycin may increase the toxicity of digoxin because antibiotics affect the liver enzymes, causing digoxin to be metabolized slower. Similarly, the simultaneous use of methotrexate and ibuprofen may result in increased methotrexate toxicity due to inhibition of kidney excretion by ibuprofen.

Similarly, drug-food interactions may occur, for example, when the drugs react with foods, supplements, or beverages including alcohol. Some of the drugs may interfere with the body's ability to absorb nutrients. In the same way, certain herbs and food may lessen or increase the impact of a drug. Likewise, drug-disease interactions may occur, for example, when a drug worsens or exacerbates an existing medical condition. For instance, an OTC nasal decongestant containing pseudoephedrine may increase blood pressure and thus may be required to be avoided by hypertensive patients.

To collate and make available interaction information relating to side effects, such as potential drug interactions between the prescribed drug(s) and commonly consumed food, supplements as well as an additional drug(s) along with other relevant information, such as therapeutic and dosing details of the drug(s) prescribed to patients, the drug information aggregation system 102 may communicatively couple to a plurality of sources of publicly and privately accessible information in the network environment 100.

In an example embodiment, the drug information aggregation system 102 may primarily interact with two types of information sources to access data for its operation. The first information source may comprise a source of health data of patients, for instance, data servers and/or databases implemented and/or maintained by one or more healthcare plan providers containing the health data of patients hereinafter referred to as plan servers 120-1 . . . 120-n. The second type of information source may comprise a variety of sources of information pertaining to topics related to drugs, diseases, health, wellness, medical science, research, and so on, containing information relating to drug approval, education, drug's mechanism-of-action, emerging trends information, and drug history information, for instance. The sources of the second type of information may be publicly and privately maintained databases and servers etc. The publicly and privately maintained servers are hereinafter referred to as one or more source servers 114-1 . . . 114-n.

In the network environment 100, in an example, the one or more source servers 114-1 . . . 114-n and one or more plan servers 120-1 . . . 120-n communicatively coupled with the drug information aggregation system 102 via the same or different communication means.

The one or more source servers 114-1 . . . 114-n and the one or more plan servers 120-1 . . . 120-n may enable storing and accessing data and may be accessed by the drug information aggregation system 102 via a network 116 for retrieving their respective data. The one or more source servers 114-1 . . . 114-n and one or more plan servers 120-1 . . . 120-n may be implemented as any of a variety of conventional computing devices, including, a desktop, a personal computer, a notebook or portable computer, a workstation, a mainframe computer, and a laptop. Further, in one example, the one or more source servers 114-1 . . . 114-n and one or more plan servers 120-1 . . . 120-n may be a distributed or centralized network system in which different computing devices may host one or more of the hardware or software components of the one or more source servers 114-1 . . . 114-n and one or more plan servers 120-1 . . . 120-n.

For example, network 116 may be a single network or a combination of multiple networks and may use a variety of different communication protocols. Network 116 may be a wireless or a wired network, or a combination thereof. Examples of such individual networks include, but are not limited to, Global System for Mobile Communication (GSM) network, Universal Mobile Telecommunications System (UMTS) network, Personal Communications Service (PCS) network, Time Division Multiple Access (TDMA) network, Code Division Multiple Access (CDMA) network, Next Generation Network (NGN), Public Switched Telephone Network (PSTN). Depending on the technology, network 116 may include various network entities, such as gateways, and routers; however, such details have been omitted for sake of brevity of the present description.

In accordance with an example implementation of the present subject matter, the drug information aggregation system 102 comprises a communication module 104. The communication module 104 accesses the health data of patients who are enrolled in healthcare plans including federal, national, state health organizations, government-funded, and employer-funded healthcare plans. In an example, the communication module 104 may access the health data of patients, from one or more plan servers 120-1 . . . 120-n, over the network 116, wherein the one or more plan servers 120-1 . . . 120-n may belong to providers of the healthcare plans of the patients. The health data of a patient comprises data pertaining healthcare plan of the patient and may include clinical demographic data, such as age, gender, race, region, weight, height, state of residence, medical condition(s) if any of the patient, etc. The health data also includes information relating to one or more drugs prescribed to the patient and known/recorded OTC drugs and supplements being taken. Those skilled in the art will appreciate that a patient may be enrolled in more than one healthcare plan that may be individually tailored for a particular patient's history, symptoms, and diagnoses for one or more than one medical conditions. In such cases, the health data relating to each such plan may be retrieved by the communication module 104.

A healthcare plan may include multiple complementary prescriptions for treatment for a particular medical condition. In an example implementation, the communication module 104 may access the health data of the patients from servers maintaining data pertaining to the healthcare plans subscribed by the patients. In an example, the communication module 104 may communicate with the one or more plan servers 120-1 . . . 120-n of the healthcare plans, over the network 116, for patients to confirm and, if applicable, update the health data with their medical conditions, the prescription drugs, etc.

In an example, the communication module 104 may include a variety of software and hardware interfaces that allow interaction of the drug information aggregation system 102 with other communication and computing devices, such as network entities, web servers, external repositories, and peripheral devices. The communication module 104 may call and communicate with one or more source servers 114-1 . . . 114-n and one or more plan servers 120-1 . . . 120-n through an interface, which may be an application program interface (API) in one example.

The communication module 104 is further configured to receive drug approval information relating to the drugs from prescribing information (PI) documents. The PI documents include information provided by drug regulatory authorities relating to one or more drugs and their uses. The PI documents may also include information relating to side effects that patients may expect, their occurrence rates, typical onset and duration, and steps involved for their mitigation or resolution including contacting medical professionals.

Those skilled in the art will appreciate that a PI document may contain a summary of essential scientific information and data from clinical trials needed for safe and effective use of the prescription drugs or biological products that are required by authorities for approval for use; be informative and accurate and neither promotional in tone nor false or misleading; and be updated when new information becomes available that causes labeling to become inaccurate, false, or misleading. In one example, the PI documents may be published by the drug regulatory authorities on webpages that the communication module 104 may access.

Furthermore, the communication module 104 is adapted to communicate with a plurality of preidentified web sources to obtain educational information relating to one or more drugs. For instance, the educational information may be obtained for the drugs prescribed to a patient as identified based on the health data of the patient. The educational information comprises publications relating to the drugs as well as medical conditions associated with the drugs. In an example, the educational information relating to one or more drugs may include, but not are limited to, dosages, possible drug interactions and other side effects, and contraindications along with safety and/or efficacy ratings.

In another example, the educational information relating to the medical conditions may include, but is not limited to, symptoms of the various medical conditions; causes/risk factors associated with each medical condition; the impact of various medical conditions and their progression if goes untreated; benefits of treatment compliance and disease control with suggestions to improve compliance based on the information about risks and impact of poor or non-compliance; alternative therapeutic options in cases where ongoing treatment is not able to improve patients' medical condition; and recommended non-medicine and non-supplemental steps, such as meditation, exercise, diet, nutrition, etc., that may be taken by patients to improve their health outcomes.

The communication module 104 also retrieves mechanism-of-action information relating to each of the drugs. As will be apparent to one skilled in the art, the term “mechanism of action” is a well-known pharmacological term commonly used when discussing drugs and may refer to how a drug is believed to work on a molecular level in the body. The mechanism-of-action information includes the mechanism-of-action of at least one molecule present in one or more drugs. As discussed above, “mechanism-of-action” describes the process by which a molecule, such as a drug, functions to produce a pharmacological effect. A drug's mechanism of action may refer to its effects on a biological readout such as cell growth, or its interaction and modulation of its direct biomolecular target, for example, a protein or nucleic acid.

In an example, a source for retrieving mechanism-of-action information may include but is not limited to, a description from a PI document, web copy of prior marketing/patient education material, online clinical publications, general media, disease education portals, treatment guidelines, and/or any other web source.

The communication module 104 further retrieves emerging trends information relating to the medical conditions associated with one or more drugs. The emerging trends information includes national and international clinical guidelines for managing medical conditions. In one example, the emerging trends information relating to medical conditions, (i.e. cancer) may include the use of thermal imaging technique for detection of cancerous tissue by analyzing block of pixels of exposed skin tissue in a thermal image, the use of an artificial intelligence (AI) tool to analyze the region of hotspots in a thermal image to determine whether breast tissue is cancerous or not. Other types of emerging trends may include but are not limited to, change in treatment algorithms where the order of drugs for treatment is changed based on new clinical evidence, or beneficial new indications to a drug, e.g., a newly approved indication for heart failure protection from an existing diabetes drug, etc.

The communication module 104 also retrieves drug history information relating to one or more drugs. The drug history information is indicative of information relating to development of the drug and may include drug class, name of the drug, date of approval by the drug regulatory authorities, date when the drug was first made available for public use, name of manufacturers, original manufacturer, pricing, number of clinical publications, reported side effects, etc. In an example, sources of drug history information may include but are not limited to, online business publications, online marketing, clinical publications, and publicity statements, media coverage.

In an embodiment, the information retrieved by the communication module 104 may be updated periodically or as and when new information is published on the information sources to ensure that the information that is provided to users is relevant and up-to-date.

In accordance with another example implementation of the present subject matter, the drug information aggregation system 102 comprises an expert input module 106. Once the information is retrieved from one or more of the above-mentioned sources, the information may be reviewed and revised by experts to ensure accuracy and, in some examples, simplicity of the information, thereby enabling an average patient to understand and act upon said information.

Accordingly, the expert input module 106 receives expert inputs relating to the information retrieved by the communication module 104. In an example, the expert input module 106 may be accessible to experts of an external review board who may include but are not limited to, physicians or specialists (MDs), nurse educators, pharmacists, and health literacy experts. The expert input module 106 may be accessible to experts through computing devices, herein referred to as user devices 118, that the experts may use to interact with the drug information aggregation system 102 to review the data retrieved by the communication module 104.

The process of augmenting the information retrieved by the communication module 104 using the expert input module 106 may in one example include reviewing the retrieved information by nurse educators to ensure that the retrieved information is accurate, simple, and easy to understand; vetting the information, that is passed by the nurse-educators, by the pharmacists to ensure that dosing and clinical information retrieved by the communication module 104 is accurate; vetting the information, that is passed by the pharmacists, by the appropriate MDs, based on specialty/therapeutic category, for final approval; and reviewing the information, that is passed by the MDs, by the health literacy experts, editors, and fact-checkers to ensure that the information is properly sourced and accurate and, in an example, does not violate any existing copyrights. Such experts may be able to access the expert input module 106 on an as-needed basis, or a regular basis. Also, in an example, to ensure the privacy of the patients, the experts may not have access to any kind of information about the patient's health status, regular daily activities, or clinical demographic data.

In an example, the expert input module 106 may enable experts to create, submit, view, and search information retrieved by the communication module 104, and permit experts to create, initiate, pause, delete, and clone said information. The expert input module 106 may be accessed by a plurality of experts to process the retrieved information individually or collaboratively.

The expert input module 106 may include, for example, any type of operating system and/or application program residing in the drug information aggregation system 102. The expert input module 106 may be accessed through network 116 by browsers or locally installed client applications on at least one user device 118. Examples of the user device 118 may include but are not limited to a desktop computer, a laptop computer, a tablet, a smartphone, a smart whiteboard, a pre-loaded tablet or smartphone, a pre-formatted tablet, a smartphone, and similar devices. As shown in FIG. 1, a user device 118 may be configured to receive inputs from the experts and communicate said inputs to the expert input module 106, or components thereof.

In accordance with another example implementation of the present subject matter, the drug information aggregation system 102 further comprises an information aggregation module 108 configured to aggregate the information, which is processed by the experts, in a database to provide users with personalized information. The information aggregation module 108 constructs and stores a drug information database 110. The drug information database 110 includes the information that is retrieved by the communication module 104 and processed by the experts.

In accordance with an example implementation of the present subject matter, the drug information aggregation system 102 comprises a side effect information module 112 to provide information pertaining to side effects that may occur due to consumption of the one or more drugs prescribed to the patients or due to consumption of at least one of the additional drug or supplements. The side effect information module 112 may also provide information relating to expected onset, duration, and recommended steps that may be taken to mitigate and/or manage such side effects, for example, recommending the patient to contact his/her healthcare provider first for exact directions.

In accordance with another example implementation of the present subject matter, the drug information aggregation system 102 may comprise an interaction information module 122 to provide information pertaining to the interaction that may occur when the one or more drugs prescribed to the patients are co-administered with the at least one additional drug or supplement. The interaction information module 122 indicates potential drug interactions, such as drug-drug interactions, drug-food interactions, drug-disease interactions, etc., by using information collated in the drug information database 110 relating to the drugs prescribed to the patients. For this purpose, when an indication of an additional drug or supplement, apart from the drugs that are prescribed to the patient, that a patient consumes or intends to consume, is provided to the interaction information module 122, the interaction information module 122 conducts a check for potential drug interactions. For instance, if a user, who is suffering from heart disease and diagnosed with atherosclerosis, and has been prescribed rosuvastatin as one of the drugs for the disease, was to consider co-consumption of green tea and the rosuvastatin and queries the drug information aggregation system 102, the user is provided with interaction information indicating that excessive consumption of the green tea and rosuvastatin might lead to reduced drug efficacy or risk of drug toxicity.

Based on the drug information database 110, the interaction information module 122 may also provide the drug interaction information that is indicative of a change in the action or side effects of at least one of the one or more drugs and/or additional drugs or supplement when at least one of the one or more drugs and the at least one additional drug or supplement are co-administered to a patient.

The information compiled under interaction information module 122 may include the most common side effects of each drug as indicated on the drug label, the expected onset and duration from sources including the prescribing information, and other published clinical evidence.

In one example implementation, the system 102 may not make specific health recommendations, and rather provide suggestions as to potential steps that may be taken to manage side effects. Simultaneously, the system 102 may instruct users to contact their healthcare provider or a pre-nominated medical professional from the healthcare provider overseeing that patient's care, such as a doctor, nurse, or other health experts, for advice.

In an example, the information from the side effect information module 112 as well as the interaction information module 122 is communicated to the plan servers 120-1 . . . 120-n. The plan servers 120-1 . . . 120-n may in turn send said information to their patient as well as to the medical professional overseeing that patient's care to ensure the patient is properly counseled. In an example, a patient may connect with the plan servers 120-1 . . . 120-n of his healthcare plan provider using a device similar to the user device 118. The patient may be able to access the information received from the plan servers 120-1 . . . 120-n by using said device.

Thus, by being able to identify harmful side effects caused by the drug interactions, patients' understanding of their ongoing medical treatment, drug safety awareness, continued medication adherence, etc., may be significantly enhanced. Also, adverse medical events, such as drug interactions, interruptions, or discontinuation of prescribed medication treatment, etc. may be suitably avoided. The present subject matter provides an ordered, customized, and easy-to-understand interaction and side effects information source, and thus, has the potential to positively impact the health outcomes for the patients who are on the prescribed drugs by substantially improving information distribution and management via a defined number of interventions.

This may allow patients and/or their caregivers to participate in a healthcare plan to better manage the patient's drug regimen while managing and avoiding potential drug interactions, as indicated above, and encourage them to reach out to members of their healthcare provider(s), ultimately leading to improved communications.

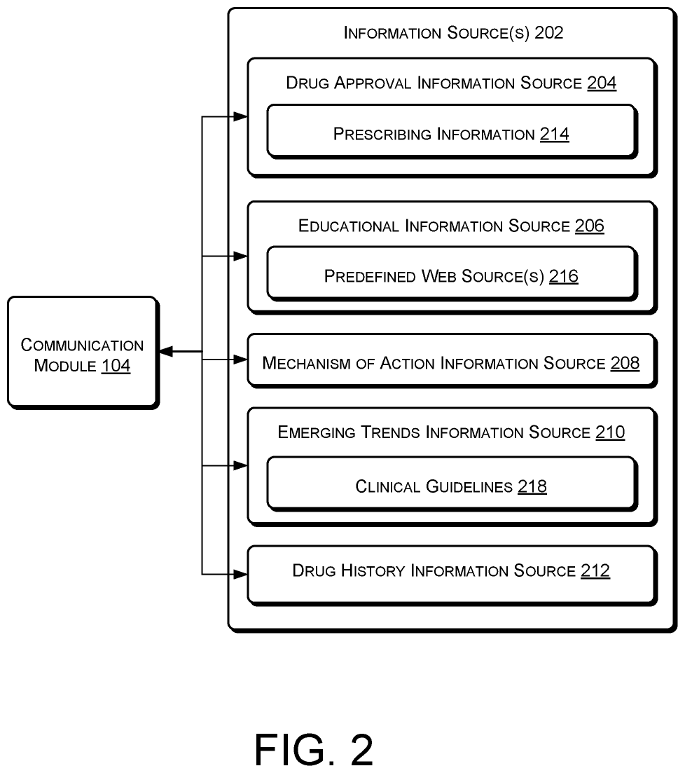

FIG. 2 depicts the communication module 104 in interaction with a variety of information source(s) 202, according to another example implementation of the present subject matter. As described previously, information from a plurality of disparate sources is collected, processed, and aggregated in order to provide meaningful drug interaction information to patients, for example, the patients who have been prescribed more than one drug under their healthcare plans.

In an embodiment, on one hand, the communication module 104 interacts with the plan servers 120-1 . . . 120-n to retrieve information relating to drugs prescribed to a patient. On the other hand, the communication module 104 interacts with the various information source(s) 202 to retrieve different types of relevant information that is processed by experts for aggregating and providing drug interaction information to the patients. In an example embodiment, the drug interaction information may be provided to the healthcare plan providers in a blinded form in accordance with the one or more drugs prescribed for the patients. The healthcare plan providers may then unblind this drug interaction information and provide the same to their patients.

In an example, the information source(s) 202 may include a drug approval information source 204, an educational information source 206, a mechanism-of-action information source 208, an emerging trends information source 210, and a drug history information source 212. The information source(s) 202, in an example, may be implemented as web servers, such as the above-mentioned one or more source servers 114-1 . . . 114-n.

In an example, the communication module 104 may interact with the drug approval information source 204 to access prescribing information 214 pertaining to the drugs as well as the supplements. The prescribing information 214 may contain, for example, a summary of essential scientific information, data from clinical trials needed for the safe and effective use of the drugs and supplements, and may be based on drug labels approved by food/drug regulatory authorities of jurisdiction and written in the patient-friendly language, and the like.

The essential scientific information, pertaining to the drugs and supplements, that are provided in the prescribing information 214 may include, for example, the standardized indications for each molecule; common side effects, rare and dangerous but observed side effects, known drug interactions, warnings, and precautions/applicable black box warnings; dosing/titration details, dose strengths, recommended do's and don'ts; potential lab abnormalities and their meaning, the importance of monitoring them; required routine clinical or lab tests and their frequency and reasons; efficacy results, expected health outcomes if the patient follows the provided instructions.

The prescribing information 214, in an example, may also include data from publications and medical literature, for each molecule of the drugs and supplements post-regulatory approval by the drug regulatory authorities both public and private. Such data, for example, may include information relating to adverse events, especially after approval by the drug regulatory authorities; changes to the drug label to account for additional side effects or safety signals, newly discovered and documented drug interactions with other prescription drugs or herbal or nutritional supplements, etc.; announcements and recalls issued by the drug regulatory authorities; safety and tolerability issues, e.g., the emergence of Stevens-Johnson syndrome, etc.; additional therapeutic benefits; off-label uses, for example, for treating other medical conditions; breaking news about the molecules, e.g., safety recalls, new uses, etc.

In another example, the communication module 104 interacts with the educational information source 206 to access educational information about drugs. The educational information source 206 may comprise predefined web source(s) 216 that include publications pertaining to the drugs and supplements and medical conditions/functions associated with said drugs and supplements. For instance, as indicated above, such publications may include information about symptoms of the various medical conditions; causes/risk factors of each medical condition; the impact of an uncontrolled medical condition and its progression if untreated or uncontrolled, and the like.

The predefined web source(s) 216, for example, may comprise information published by established internationally recognized disease/treatment organizations, associations, and government bodies. Examples of such organizations, associations, and government bodies may include but are not limited to the National Institute of Health, Centers for Disease Control and Prevention, American Heart Association, American Cancer Society, National Comprehensive Cancer Network, American Lung Association, American Diabetes Association, American Academy of Pain Medicine, American Gastroenterology Association.

The predefined web source(s) 216 may additionally include educational material on specific lifestyle recommendations related to the medical condition(s) currently being treated. These lifestyle recommendations may be prepared by publishers of the predefined web source(s) by working in consultation with pharmacists, nutritionists, dietitians, nurse-educators, accredited fitness coaches, wellness and lifestyle experts, and the like. Said lifestyle recommendations may include, for example, information on foods and nutritional supplements to avoid drugs, thereby minimizing drug interactions; appropriate and beneficial foods and supplements; avoidable foods and supplements; appropriate fitness activity suggestions: do's and don'ts; mindfulness techniques; motivational and inspirational advice to encourage compliance with therapeutic and overall health regimen, etc.

In yet another example, the communication module 104 connects with one or more mechanism-of-action information source 208 to access the mechanism-of-action of the molecules of the drugs and/or supplements. As indicated above, the mechanism of action describes how a drug or supplement is theoretically believed to work inside the body, creating effects that may lead to relief, amelioration, or control/cure of the symptoms and medical condition(s) that the drug or supplement is intended to treat/improve. Examples of mechanism-of-action information source 208 may include, but are not limited to PI documents; prior marketing/patient-education material; clinical publications; descriptions in general media, disease education websites, treatment guidelines, etc.

In another example, the communication module 104 may also obtain data on emerging trends information relating to the drugs or supplements and whenever emerging trends information is identified, the same may be communicated to the plan servers 120-1 . . . 120-n. The plan servers 120-1 . . . 120-n may then send said emerging trends information to their patient as well as to the medical professional overseeing that patient's care to ensure the patient is properly counseled in respect of said information if required. Accordingly, the communication module 104 may communicate with emerging trends information source 210 to access the emerging information relating to medical conditions/functions associated with the drugs or supplements contained in clinical guidelines 218 of the national and international stature.

The clinical guidelines 218 may provide insight into new or emerging techniques that may help manage the one or more medical conditions for which the patient is currently being treated. The clinical guidelines 218, for example, may be issued nationally and internationally by major clinical organizations, bodies, or authorities and may be created in consultation with leading clinical authorities and researchers in the respective therapeutic area. In one example, the emerging information relating to the medical conditions, such as novel coronavirus-2019, may include but are not limited to, approval of newer vaccines and treatments, booster shots for various vaccines, other new treatments approved or for use under emergency authorizations to reduce symptoms or hospitalization rates, etc.

Further, the communication module 104, in an example, may interact with the drug history information source 212 to access the history of the drug and supplement in the market. The drug history information may be helpful for patients, for example, in understanding the overall efficacy, effectiveness, and efficiency of drugs or supplements they are consuming. This historical information may aid the patients in deciding whether to consume/continue with the given drugs and supplements or not. The drug history information source 212 includes, for instance, the general history/story of the drug from research to approval to the overall experience and track record in the market, which may enable a patient to assess if the drugs and supplements that they are taking have a good track record. Often, patients do not have any information about their drugs, and knowing that the drug they are consuming has a proven track record and established safety profile may instill confidence in their medical professional's prescription decision and may improve their adherence to this regimen.

Examples of the history information of the drugs may include drug class, name, date of approval by the drug regulatory authorities, date of introduction for public use, name of manufacturers, original manufacturer, number of clinical publications, current average wholesale price (AWP) for the branded generic formulation both, etc. The drug history information source 212 may include but is not limited to business publications, marketing company statements, etc.

Once the information from the variety of information source(s) 202 is retrieved, the same is then processed to create the drug information database 110. Based on the information collected in the drug information database 110, whenever a user queries the drug information aggregation system 102 to know about potential drug interaction or other side effects, the user may be provided with drug interaction information or known or reported side effects occurrence or specific information corresponding to the user's query. The techniques of processing the retrieved information and providing the drug interaction information and the side effects information have been discussed in detail subsequently.

In an example implementation, the drug information aggregation system 102 may also respond to queries submitted by the user in the form of an image. For example, if a user provides an image of the drugs or supplements, the drug information aggregation system 102 may identify the potential drug interaction or other side effects of corresponding drugs based on the images as well. For this purpose, the communication module 104 may interact with the drug history information source 212 or any other public source, e.g., a database maintained by respective drugs or supplements manufacturers, to access image information of packaging of various forms and dosages of a plurality of known approved drugs and supplements, and extract attributes comprising one or more of size, color, markings, volume, and shape corresponding to the packaging.

The image information comprising the attributes is then stored in the drug information database 110. The sources of image information may include but are not limited to web pages of third-party vendors, drug and supplement manufacturers, Food and Drug Administration (FDA), Canadian Food Inspection Agency (CFIA), European Medicines Agency (EMA), and the like. The drug may be identified by comparing attributes of the image of the drug provided by the user to the attributes of the various drugs stored in the drug information database 110. Once the drug is identified, the information aggregation system 102 may provide information relating to the identified drug retrieved from the various source servers as well as the side effects or the potential drug interaction the identified drug may have with other commonly consumed food, drugs, and supplements.

The information aggregation system 102 thus may help mitigate risks, such as the use of wrong dosage strength or wrong medicine, excessive drug dosage, prolonged duration of use, drug interactions, and polypharmacy by providing the patients with customized education, advice, and support in simple, easy-to-understand language to meet their medical education needs.

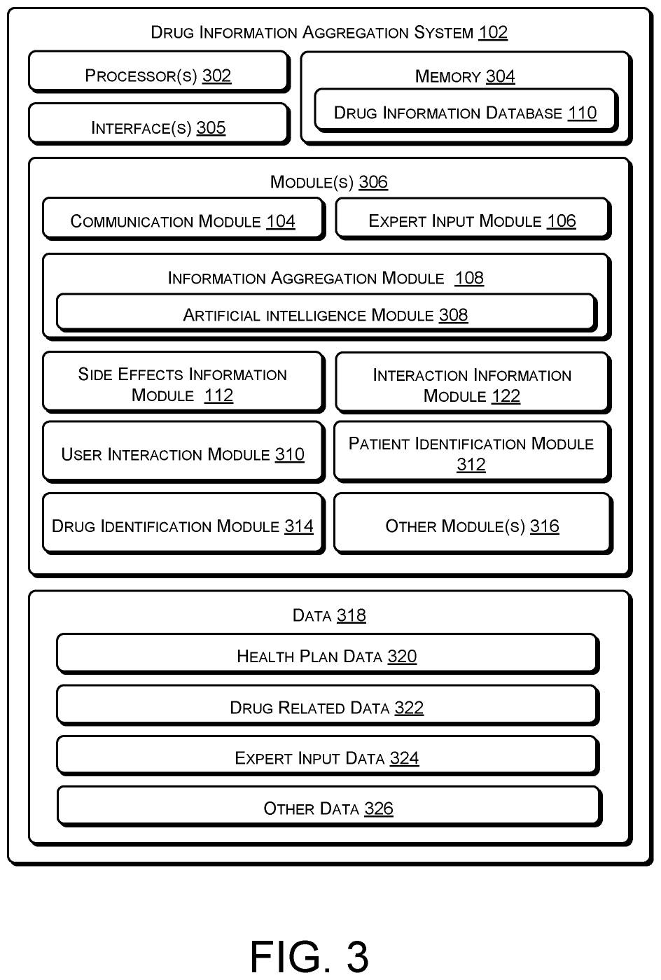

FIG. 3 illustrates the drug information aggregation system 102 according to an example implementation of the present subject matter. In an example, the drug information aggregation system 102 depicted in FIG. 3 may be a server that is external to the plan servers 120-1 . . . 120-n of the healthcare plan providers. For example, the drug information aggregation system 102 may be connected to one or more plan servers 120-1 . . . 120-n via the network 116. In another example, the drug information aggregation system 102 may also be part of a hosted service executed on one or more of the plan servers 120-1 . . . 120-n. In the example implementation depicted in FIG. 3, the drug information aggregation system 102 is an external system communicatively coupled to one or more plan servers 120-1 . . . 120-n.

As explained previously, in an example, the drug information aggregation system 102 may access the highly specific, condition-related proprietary health data, secured in the plan servers 120-1 . . . 120-n of the healthcare plan providers of patients, to offer patients a system for identifying the possible drug interactions or other side effects while complying with health and general privacy standards, such as Health Insurance Portability and Accountability Act (HIPAA), Personal Informational Protection and Electronic Documents Act (PI PEDA), General Data Protection Regulation (GDPR), etc.

The drug information aggregation system 102, in one example implementation, in compliance with the health and general privacy standards, may not release information to anyone but the patient and their healthcare plan provider per the usage agreement between the healthcare plan provider and the drug information aggregation system 102, and the patient and drug information aggregation system 102.

In an example implementation, the drug information aggregation system 102 may not have direct access to the patient's data. While enrolling in the system 102, each healthcare plan provider may be asked to assign a unique multi-digit code for each of their patients to mask/anonymize the identity of the patient and preserve their data privacy. This unique number may then be shared with the system 102 and may thus become the patient's default identity till the time they are associated with the system 102. All customized information for each patient may be sent to the patient's healthcare plan provider which may then de-anonymize and disseminate it to their patients.

The drug information aggregation system 102 of the present subject matter provides ongoing customized education, advice, and support in simple, easy-to-understand language to meet the medical education needs of the patients who require a single source of easily accessible and reliable information to identify side effects or drug interactions associated with the consumption of prescribed or non-prescribed drugs, food, supplements, diseases, etc.

As depicted in FIG. 3, in an example implementation, the drug information aggregation system 102 may include at least one processor 302 and a memory 304 coupled to the processor 302. In an example, the processor 302 may be implemented as microprocessors, microcomputers, microcontrollers, digital signal processors, central processing units, state machines, logic circuitries, and/or any devices that manipulate signals based on operational instructions. The memory 304 may include any computer-readable medium known in the art including, for example, volatile memory (e.g., RAM), and/or non-volatile memory (e.g., EPROM, flash memory, etc.). The memory 304 may also be an external memory unit, such as a flash drive, a compact disk drive, an external hard disk drive, or the like. The drug information aggregation system 102 may comprise module(s) 306 and data 318 coupled to the processor 302. In one example, the module(s) 306 and data 318 may reside in memory 304.

The module(s) 306 may include routines, programs, objects, components, data structures, and the like, which perform particular tasks or implement particular abstract data types. The module(s) 306 may further include modules that supplement applications on the drug information aggregation system 102, for example, modules of an operating system. The module(s) 306 further includes modules that implement certain functionalities of the drug information aggregation system 102, such as processing the information accessed by the drug information aggregation system 102. The data 318 serves, amongst other things, as a repository for storing data that may be fetched, processed, received, or generated by one or more of the module(s) 306.

In an example, data 318 may comprise a health plan data 320, wherein the health plan data 320 is fetched by the drug information aggregation system 102 from the one or more plan servers 120-1 . . . 120-n. The data 318 may also comprise drug related data 322, wherein the drug related data 322 is fetched by the drug information aggregation system 102 from the one or more source servers 114-1 . . . 114-n. Data 318 may further comprise an expert input data 324, wherein the expert input data 324 is received from external users interacting with the information aggregation system 102 to review the drug related data 322 and other data 326.

In accordance with an example implementation of the present subject matter, module(s) 306 may include a user interaction module 310, a patient identification module 312, a drug identification module 314, and other module(s) 316, in addition to the communication module 104, expert input module 106, information aggregation module 108 and interaction information module 122, described previously. The other module(s) 316 may include programs or coded instructions that supplement applications and functions, for example, programs in the operating system of the drug information aggregation system 102.

Also, as depicted in FIG. 3, in an example implementation, interface(s) 305 may be coupled to the processor 302. The interface(s) 305 may include a variety of software and hardware interfaces that allow interaction of the drug information aggregation system 102 with other communication and computing devices, such as network entities, external repositories, and peripheral devices. The interface(s) 305 may also enable the coupling of components of the drug information aggregation system 102 with each other. Further, in an example, the interface(s) 305 may couple the user device 118 to the system 102.

The communication module 104, in an example, may also include a variety of software and hardware interfaces that allow interaction of the drug information aggregation system 102 with other devices, such as network entities, web servers, and external repositories, and peripheral devices. For example, the communication module 104 may couple the drug information aggregation system 102 with the one or more source servers 114-1 . . . 114-n, plan servers 120-1 . . . 120-n, and the user device 118.

In operation, the communication module 104 interacts with the source servers 114-1 . . . 114-n to retrieve the drug approval information, the educational information, side effects occurrence, onset, duration and management information, the mechanism-of-action information, the emerging trends information relating, the drug history information, and the image information, respectively, as discussed previously. The retrieved information is stored in memory 304 as the drug related data 322.

As discussed previously, the information retrieved from the various sources may require to be meaningful for it to be presented to the patients, and therefore, experts' input is needed for processing the retrieved information to make it meaningful, for instance, by verifying its accuracy.

Accordingly, as discussed previously, the experts from the members of the external review board may use their respective user devices 118 to interact with the drug information aggregation system 102. The expert input module 106 allows the experts to access the information that is retrieved by the communication module 104. The experts access said information to review and provide their inputs on the same using the user device 118 to process the retrieved information. The expert inputs ensure the scientific accuracy as well as simplicity of the information.

In an example, the expert input module 106 may allow a variety of information to be reviewed by the experts, such as tables, legends, citations, indexing, proofreading, etc. In another example, the expert input module 106 may provide controls for selecting, and modifying various aspects of the retrieved information, such as formatting, grammatical or stylistic corrections, and even word replacements through synonym/antonym suggestions. In yet another example, to enable collaborative authoring, the expert input module 106, may track activities done by the experts and provide push notifications regarding changes that may affect the work done by a particular expert. Similarly, co-experts may be notified about changes, versions, and/or notes that may be restored and/or compared, and a pivot history may be provided.

To bring the information reviewed by the experts together and convey it accurately, the information may require aggregation. Accordingly, as discussed previously, the information aggregation module 108 aggregates the information which is processed by the experts in the drug information database 110 to provide users with personalized information. The process of aggregation of the information may include information indexing, tagging, etc., to organize the experts' inputs. In one embodiment, the information aggregation module 108 may include an artificial intelligence (AI) module 308 that may be pre-trained to identify and flag newly published information pertaining to each of the drugs being covered for further review. This information is forwarded to the expert input module 106 where it may be tagged and reviewed first by the experts. Once vetted, the same may be re-written in simple language by a patient communications expert, then edited and proof-checked, and reverified for accuracy and simplicity by the experts.

In one embodiment, the AI module 308 may use a natural language processing model to construct the drug information database 110 based on the information reviewed by the experts. In an operation, the AI module 308 may also constantly interact with the plurality of publicly and privately accessible information sources for breaking news relating to the one or more drugs, medical conditions associated with the one or more drugs, new medical treatments, new information on medical conditions, etc., to help construct the drug information database 110. Once the drug information database 110 is constructed, it is stored inside the memory 304. In some embodiments, the drug information database 110 may be external to the drug information aggregation system 102, and the drug information aggregation system 102 may communicate wirelessly or via a wired connection therewith.

In an operation, when the indication of a drug prescribed to the patient, or additional drug, or supplement that a patient consumes or intends to consume is provided to the side effect information module 112, the side effect information module 112 conducts a check for a potential side effect(s) of based on the information stored in the drug information database 110. Based on the checking, the side effect information module 112 returns side effects information pertinent to the prescribed drug, or additional drug, or supplement that the patient consumes or intends to consume.

In one example implementation, there may be a separate module for identifying drug interactions that may be caused by co-consumption of the prescribed drug and additional drug, or supplement. Accordingly, when the indication of the additional drug or supplement that the patient consumes or intends to consume, apart from the drugs that are prescribed to the patient, is provided to the interaction information module 122, the interaction information module 122 conducts a check for potential drug interactions based on the information stored in the drug information database 110. Based on the checking, the interaction information module 122 returns drug interaction information pertinent to the additional drug or supplement. The patient is then advised to contact their healthcare provider team for specific advice.

In one example implementation, the side effects information, and the drug interaction information may be provided to the one or more plan servers 120-1 . . . .120-n of the healthcare plan providers to which the patient is subscribed. Thereafter, the plan servers 120-1 . . . .120-n may de-anonymize the side effects information and the drug interaction information and disseminate this information along with other health plan-related details to the users via the mode of their preference, e.g., email, mail, text, phone, videos, or application on smartphone/tablet. In another example embodiment, it is also possible that a user who is not a subscriber of any of the healthcare plans may directly connect with the drug information aggregation system 102 by simply entering, via the user device 118, the details of two or more drugs, OTCs, or supplements to check for the drug interactions.

Accordingly, the side effect information module 112 and the drug interaction information module 122, respectively, may be able to provide customized and personalized side effects and drug interaction information to patients and/or authorized caregivers through their healthcare plan based on where they are on their treatment journey. The patients who initiate new or additional treatments may receive step-by-step guidance to help them fully comply with the new drug regimen and improve adherence to other drugs.

In an example, side effects information may include a description of an alternate course of action according to the determined type of side effect. Similarly, the drug interaction information may include a description of an alternate course of action according to the determined type of interaction. An example alternate course of action may involve looking up for an alternate drug in the same drug class or type as the additional drug or supplement. For example, if the side effect information module 112 identifies based on the patient's query that the patient is likely to have an adverse effect upon consuming Favipiravir, the side effect information module 112 may suggest contacting the patient's healthcare provider.

In another example, if the interaction information module 122 identifies that, due to a genetic mutation in a patient and based on adverse event data, the patient will likely experience an adverse event upon consuming Favipiravir, the interaction information module 122 may repeat the analysis for Oseltamivir, another anti-viral drug. If the interaction information module 122 determines that the Oseltamivir, an alternate drug, may induce an adverse effect in the patient, the interaction information module 122 will inform the patient that there may be potential risk and therefore recommend that they discuss this issue with their healthcare provider who may recommend alternative drugs. In instances where patient directly signs up with the drug information aggregation system 102, the drug information aggregation system 102 will inform them of the facts and potential alternate options that they should discuss with their healthcare provider for the viability of Oseltamivir as a potential alternate drug.

Thus, the interaction information module 122 may automatically recommend potential alternative drugs or approaches for the patient to discuss with their healthcare provider. Those skilled in the art should appreciate that the interaction information module 122 may suggest other alternative courses of action to the patients as well, for example, alternate timings for taking a prescription drug and additional drug and supplement, alternate dosing of either the prescription drug or the additional drug and supplement, etc., to avoid interactions. The drug interaction information, in an example, may be provided to the users as a graphical representation of the potential interaction between the one or more drugs and the at least one additional drug or supplement.

Since in almost all cases, the decision to prescribe drugs lies only with the patient's healthcare provider, if a potential drug interaction is flagged by the interaction information module 122, then the patient may be prompted to contact their healthcare provider immediately. Since healthcare plan providers will have the same information, they may decide to let the patient's healthcare provider be aware of this situation so the healthcare provider may proactively address it before the patient can bring it up, thus potentially avoiding a risky health situation. Additionally, in cases where the patient does not act on the drug interaction flag by choosing not to acknowledge the same, the drug information aggregation system 102 may also send a notification to the healthcare plan provider that the patient has been flagged about potential drug interaction, and thereafter, the healthcare plan provider of the patient may choose to send such information to the patient's healthcare provider to resolve such situation.

To access the side effects information or the drug interaction information, the users may query the drug information aggregation system 102 and may accordingly be provided with the relevant side effects or drug interaction information as per users' query. The users querying the drug information aggregation system 102 may be enrolled in one or more healthcare plans. In some embodiments, as discussed previously, the users may also query the drug information aggregation system 102 without having to be a subscriber of any of the healthcare plans. For instance, the initial data required for creating the drug information database 110 may be based on the health plan data 320 obtained from the plan servers 120-1 . . . 120n of a predefined number of users and the relevant drug related data 322. However, having built the drug information database 110 based on the health plan data 320 and the drug related data 322, the drug information aggregation system 102 may cater to other users as well, for example, users who may not subscribe to a healthcare plan or users who are newly subscribed to healthcare plans. As will be apparent, the drug information database 110 may be created based on the health plan data 320 of a certain predefined number of users and be expanded on an ongoing basis based on the health data of more users.

Accordingly, in an example embodiment, the drug information aggregation system 102 may include the patient identification module 312 that identifies patients who regularly consume one or more prescription drugs for chronic, terminal, or long-term disease/conditions and may potentially benefit from the drug information aggregation system 102.

The patient identification module 312 may communicate with the plan servers 120-1 . . . 120-n to identify one or more patients who are registered with at least one of the healthcare plans and are prescribed more than a threshold number of drugs. In some embodiments, the patient identification module 312 may also accept direct inputs from the users through the user device 118 who are willing to access the drug information aggregation system 102 and are not subscribed to any of the healthcare plans. Upon identifying the patients, the patient identification module 312 may generate an enrollment request for each of the one or more patients to register them with the information aggregation system 102. Once the enrollment request is accepted, the patient identification module 312 may send them prompts to set up their user password, select a preferred language and mode of communication from a group of email, mail, text, phone, call, and push notification, and assign a nominee caregiver, if necessary, wherein the caregivers will only be able to see the information compliant with HIPAA/Global privacy regulations.

In an example, patients, subscribed to the drug information aggregation system 102, who switch their existing healthcare plan provider will be able to ensure the portability of their health data to their new healthcare plan provider using the unique identity provided by the patient identification module 312. In addition, the users who are querying the drug information aggregation system 102 from outside the healthcare plan, may be provided either with the side effect information or the drug interaction information or both directly either via the user device 118 or through the mode of their preference.

After the completion of the enrollment process, the user interaction module 310 may interact with the user device 118 on an ongoing basis through the network 116 to receive information from a user pertaining to the patients' current lifestyle, level of fitness activity, additional drugs, and supplement usage. In some embodiments, although the users may be able to access the drug interaction information even if they are not enrolled in the drug information aggregation system 102, the users who are enrolled may have more efficient access to the drug information aggregation system 102 compared to the non-enrolled users. For example, the users who are enrolled may be required to provide information pertaining to the patient's current lifestyle, level of fitness activity, additional drug, and supplements usage only when there is a need to update such information in order to ensure accurate information on supplements, etc., in order to provide highly relevant and personalized information as compared to those users who may not be enrolled and may be required to provide such information at every instance of querying the drug information aggregation system 102.

In another embodiment, patients willing to enroll with the drug information aggregation system 102 may not already have an enrollment of any of the healthcare plan providers. In such situations, they may directly enroll with the system 102, wherein each patient may be provided with a secure multiple digits unique login identity number that may be set by the patient identification module 312. They may use the given login identity number to access the drug information aggregation system 102 to avail of drug education by indicating to the system 102 the drug and supplements they consume.

In an example embodiment, the drug information aggregation system 102 may enable the drug interaction information to be provided in a language of the user's choice. While enrolling the users to the drug information aggregation system 102, the patient identification module 312 may receive and store in the memory 304 a record that associates a language choice with a particular user. When the user logs on to the drug information aggregation system 102, the interaction information module 122 may look up the user's language choice based on the login credentials, and, accordingly, provide customized and personalized drug interaction information to patients and/or authorized caregivers in the language of user's choice. Though the users of the system 102 may be presented with an option of a range of languages to choose from, a default language, for example, English may also be set.

Language conversion information, such as a language resource pack, may be used to convert the symbolic information in data records into the user's chosen language, in an example.

The user may specify an explicit language choice, or the user's choice of language may be inferred from the language of input that the user issues into the patient identification module 312 at the time of enrollment to the drug information aggregation system 102. When the patient identification module 312 has ascertained the user's language choice, the patient identification module 312 may store an association between the user and the language choice, so that choice may be used for future requests from the same user. This would allow the users to have access to the drug information aggregation system 102 based on their native language. In some embodiments, the text of the drug interaction and side-effect information may be converted into speech for audible presentation to the user as a response to the user's query for the drug interaction information using any known text-to-speech conversion techniques. The conversion of the drug interaction and side-effect information from the text to speech may help users who cannot use their eyes or arms. Apart from both the visually impaired and physically disabled, the conversion of text to speech may also help the speech impaired and the hearing impaired, as well as the elderly people.

In some cases, the users of the drug information aggregation system 102 may want to in an example embodiment, the drug information aggregation system 102 may include a drug identification module 314 that enables the identification of a drug so as to overcome the difficulties in finding reliable information on drug prices, adverse drug reactions, pharmacology, and pharmacogenetics, or availability. To perform the identification of a drug, the drug identification module 314 may electronically receive an image of the drug to be identified from the user through the user device 118 and, based on the received image, extract attributes of the image. The attributes of the image may comprise size, color, marking, volume, and shape. Thereafter, the drug identification module 314 may identify the drug based on the comparison of attributes of the received image of the drug with the attributes of the various drugs stored in the drug information database 110 to indicate a brand name of the drug to the user. The drug identification module 314, in an example, may also indicate alternate brand names associated with the drug to be identified.

In another example embodiment, a set of templates may be provided for healthcare plan providers to insert their specific branding and colors and provide the customized information to their subscriber patients. Moreover, material to promote patient sign-ups to the drug information aggregation system 102 may also be provided.

Though patients/users of the present system 102 may come through healthcare plan providers and their patients, the health data of patients given by the plan may not be fully accurate because many patients see other medical professionals who may be outside of the healthcare plan provider network and thus on different healthcare plan provider network.

Moreover, patients may not accurately remember what drugs they were prescribed by out-of-healthcare plan medical professionals when asked by their healthcare plan-affiliated medical professional.

In such scenarios, in an example embodiment of the present subject matter, the system 102 may make use of the user interaction module 310 to prompt the patient to confirm if the patient has consulted an out-of-healthcare plan medical professional, and if so, prompt to enquire about nature of the consultation and recommended drug and its dosage by said medical professional. This may enable the healthcare plan provider to accordingly let the patient's medical professional/healthcare team know, and they may follow up and ensure that the health data of the patient on their end is updated.

An example scenario is explained herein to elaborate on the working of the system 102. In an example, the user interaction module 310, through a periodic prompt-based interaction, may receive information from an enrolled patient pertaining to their current lifestyle, level of fitness activity, additional drugs, and supplement usage. During such information interrogation, whenever the user interaction module 310 identifies that an additional drug has been prescribed to the patient, the user interaction module 310 may log such information with system memory 304 which can be subsequently used for providing the drug interaction information. One such example of information logging into the system 102 may include informing the system 102 that the patient, a white male of 50 years of age, weighing 193 lbs, with controlled type 2 diabetes (HbA1c of 7.1%) and family history of cardiovascular disease, currently taking metformin 2000 mg daily, most recently has been prescribed atorvastatin 40 mg because low-density lipoprotein (LDL) is high at 145 mg/dL.

Patients may be provided with a series of prompts, e.g., “are you currently taking erythromycin for an infection?”, or “Do you consume 2× cups of grapefruit juice a day?”. Based on their reply to the prompts, as discussed above, the interaction information module 122 may notify the healthcare plan provider or the patient directly in cases where enrollment in the system 102 is without the healthcare plan, regarding the potential drug interactions, such as drug-drug interactions, drug-food interactions, drug-disease interactions, etc., if any.

To help patients avoid potential drug interactions, the interaction information module 122 may also recommend rules of behavior for patients, such as avoiding supplements such as grapefruit juice, green tea, etc., as well as other drugs/supplements known to cause drug interactions and interfere with absorption of the prescription drugs, such as antivirals, antifungals.

In another example, the side effect information module 112 may also provide information and prompts relating to the most common side effects, such as muscle weakness, soreness, aches, stiffness, or cramps, that range from mildly irritating to intolerable as well as dark-colored, brownish urine. If so, the patients may be notified to contact the medical professional/healthcare provider immediately and seek advice. The patients may also be advised not to wait or stop continuing the drug on their own before getting medical advice; on the need for periodic liver function tests; that atorvastatin may increase their HbA1c level, for example.

In another example, the user interaction module 310 may also provide the patient with weekly reminders to take prescribed drugs; contact the healthcare provider if the patient has any symptoms of a drug interaction. The patients, through the user interaction module 310, may also be informed of other potential treatment options that the patient's medical professional/healthcare provider may consider educating and motivate them that other drug options are available to control their high existing medical condition.

In certain embodiments, the patients may be reminded periodically by the system 102, for example after the first 3 or 6 months, for making use of the system 102 to check for potential drug interactions and their side effects that may appear at any moment. Also, in certain embodiments, the patients may be prompted periodically by the system 102, for example after the first 3 or 6 months, to ask them about treatment experience on the ongoing drug, such as atorvastatin. Further, in some embodiments, the system 102, when the patient goes in for a first lipid test after treatment, may send his previous and current drug regimen compliance score to emphasize the importance of compliance and the benefits of adherence on overall health.

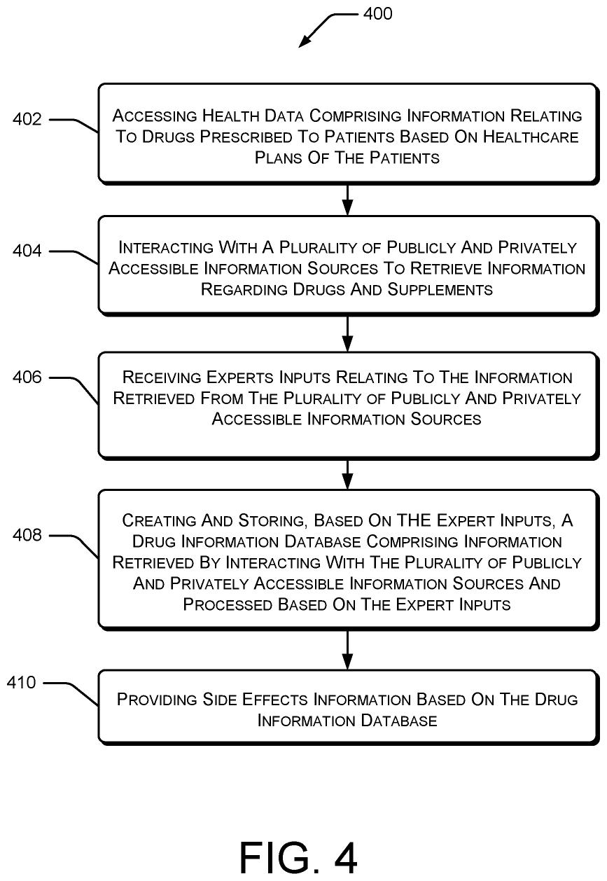

FIG. 4 illustrates a method 400 for drug information aggregation, according to an example of the present subject matter.

The order in which the method 400 is described is not intended to be construed as a limitation, and any number of the described method blocks may be combined in any order to implement the method 400, or an alternative method.

It may be understood that blocks of the method 400 may be performed, for example, by the above-described drug information aggregation system 102, as illustrated in FIGS. 1, 2, and 3. In an example, the drug information aggregation system 102 may be installed in a network environment, such as the network environment 100 described in reference to FIG.1.

The blocks of the method 400 may be executed based on instructions stored in a non-transitory computer-readable medium, as will be readily understood. The non-transitory computer-readable medium may include, for example, digital memories, magnetic storage media, such as magnetic disks and magnetic tapes, hard drives, or optically readable digital data storage media.

Referring to FIG. 4, at block 402, the communication module 104 accesses health data of patients by interacting with the one or more plan servers 120-1 . . . 120-n of their healthcare plan providers. The health data of a patient includes clinical demographic data, wherein the clinical demographic data includes information relating to one or more drugs prescribed to the patient.

At block 404, the communication module 104 interacts with a plurality of publicly and privately accessible information sources to retrieve relevant information, such as drug approval information, educational information, mechanism-of-action information, emerging trends information, and drug history information, respectively. As discussed previously, the drug approval information may be retrieved from the drug approval information source 204 which comprises prescribing information 214 pertaining to the drugs as well as the supplements. The prescribing information 214 may cover information pertaining to the efficacy, safety, tolerability, key side effects, and required lab tests if any, as well as dosing and titration for each drug and supplement.

Similarly, the educational information may be retrieved from the educational information source 206 and may include publications pertaining to the drugs, supplements, and the medical conditions/functions associated with said drugs and the supplements. In an example, the educational information may additionally include a collection of lifestyle recommendations and advice from recognized sources based on the specific medical condition and the drugs a patient takes for treating/controlling said medical condition. The lifestyle recommendations may include advice on fitness, diet, nutritional supplements, and direction for patients to consult with their dietitians/nutritionists or fitness trainers if needed. The mechanism-of-action information may be retrieved from the mechanism-of-action information source 208. The mechanism-of-action information provides a scientific explanation of how each molecule of the drugs and the supplements theoretically works inside the body, creating effects that lead to relief, amelioration, or control/cure of the medical condition that the drugs and the supplements are intended to treat/improve. The emerging trends information may be retrieved from the emerging trends information source 210 which may provide information relating to medical conditions/functions associated with the drugs or supplements.

Likewise, the drug history information may be retrieved from the drug history information source 212, wherein the drug history information may cover the general history/story of the drug and supplements to give the patient confidence that the drugs and supplements that they are taking have a track record and engender confidence in their medical professional's decision to prescribe it.

At block 406, the expert input module 106 enables vetting of the information that is retrieved by the communication module 104 from the plurality of publicly and privately accessible information sources. The vetting of the retrieved information is done by an external review body comprised of experts in medicine, nursing, pharmacology/pharmacy, and patient healthcare communications to ensure scientific accuracy as well as simplicity of the retrieved information so that it can be understood and acted upon by an average patient.

At block 408, based on the vetted information, the drug information database 110 is created and stored by the information aggregation module 108. The drug information database 110 comprises information retrieved from the plurality of publicly and privately accessible information sources and processed by the external review body.

At block 410, the side effect information module 112 provides side effects information based on the drug information database 110, wherein the side effects information may be specific and customized to where the user is on their treatment journey—a new start, first 3 months, second 3 months and so on, for example. As discussed previously, the side effects information is indicative of side effects caused by the consumption of the at least one of the one or more drugs, food, beverage, supplement, or additional drug.

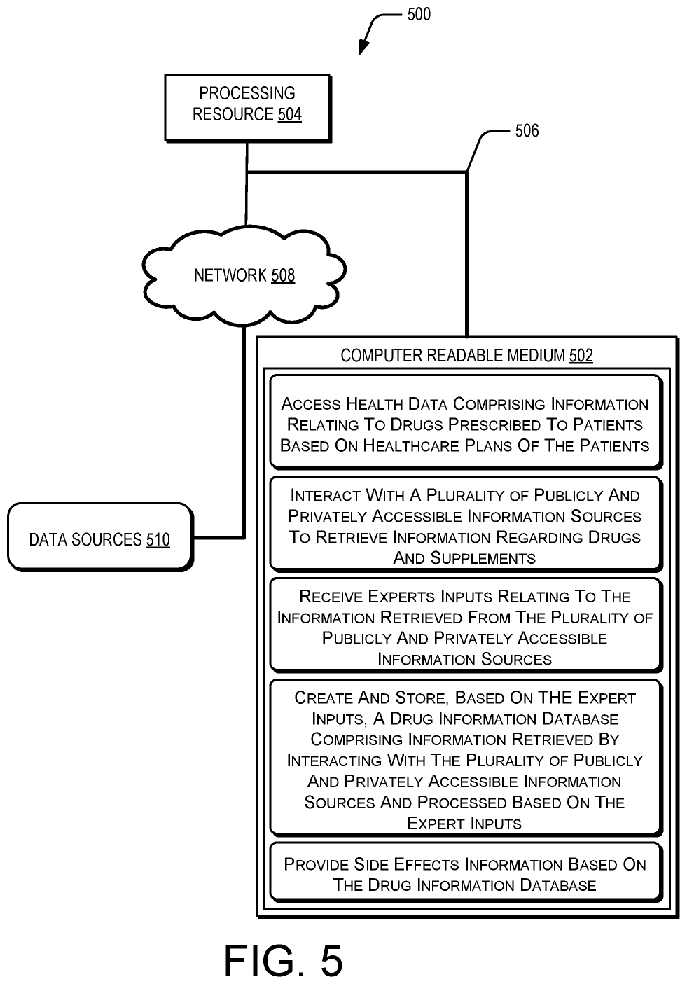

FIG. 5 illustrates a computing environment 500 implementing a non-transitory computer-readable medium 502 for aggregating drug information within the networked environment 100, according to an example of the present subject matter.

In one exemplary implementation, the computing environment 500 may include a processing resource 504 communicatively coupled to a non-transitory computer-readable medium 502 through a communication link 506. In an example, the processing resource 504 fetches and executes computer-readable instructions from the non-transitory computer-readable medium 502.

The non-transitory computer-readable medium 502 may be, for example, an internal memory device or an external memory device. In an example, the communication link 506 may be a direct communication link, such as any memory read/write interface. In another example, the communication link 506 may be an indirect communication link, such as a network interface. In such a case, the processing resource 504 may access the non-transitory computer-readable medium 502 through a network 508. The network 508 may be a single network or a combination of multiple networks and may use a variety of different communication protocols.

The processing resource 504 and the non-transitory computer-readable medium 502 may also be communicatively coupled to data sources 510. The data source(s) 510 may be used to store details, such as respective device identifiers and locations of the client devices located within the networked environment 100, in an example.

In an example, the non-transitory computer-readable medium 502 comprises executable instructions for accessing the health data of the patients based on their healthcare plans of the patients. As discussed previously, the health data of a patient may comprise clinical demographic data and the clinical demographic data may comprise information relating to one or more drugs prescribed to the patient. For example, the non-transitory computer-readable medium 502 may comprise instructions executable to implement the previously described modules.

In an example, the executable instructions may cause the processing resource 504 to interact with a plurality of publicly and privately accessible information sources to retrieve information regarding drugs and supplements within the networked environment 100. The information sources have already been described above.

Further, the executable instructions may also cause the processing resource 504 to receive expert inputs relating to the information retrieved by interacting with the plurality of publicly and privately accessible information sources. As apparent from the previous description, retrieving information regarding drugs and supplements within the networked environment 100 helps experts to process the information retrieved by the communication module within the networked environment 100.

Upon receiving expert inputs with respect to the plurality of publicly and privately accessible information sources, the executable instructions may also cause the processing resource 504 to create and store, based on the expert inputs, the drug information database that comprises information retrieved by the communication module 104 and processed based on the expert inputs within the networked environment 100.

The executable instructions may further cause the processing resource 504 to interact with the drug information database 110 to receive and provide side effects information to the patient. As apparent from the previous description, the side effects information may be indicative of side effects of at least one of the one or more drugs.

The methods and devices of the present subject matter offer techniques providing patients with the drug interaction and relevant information, disease education, and other side effects education that may lead to an improved understanding of the medical condition and to a better patient understanding of how to manage their care, and hence, improve self-advocacy and compliance to a recommended treatment plan. Importantly, it may lead to patients being more likely to respond better to their treatment plan with fewer complications and eventually the more effective use of medical services with fewer unnecessary phone calls and/or visits.

The side effects and drug interaction education program may help reinforce and support understanding of the condition of how the drug works and eventually help improve adherence and overall health of the patient. This program may demonstrate improved outcomes in a wide variety of medical conditions through individual and group support and multidisciplinary health care team coaching. The underlying objective of the drug information aggregation system is to help patients better manage their disease and complex drug regimens, improve drug adherence, and reduce complications and related costs.

1. A drug information aggregation system comprising:
at least one processor;
a memory in communication with the at least one processor;
a communication module coupled to the at least one processor, wherein the communication module is to:
access health data of a patient based on one or more healthcare plans of the patient, the health data of the patient comprising clinical demographic data, wherein the clinical demographic data comprises information relating to one or more drugs prescribed to the patient, and
interact with a plurality of publicly and privately accessible information sources to retrieve at least one of:
drug approval information relating to the one or more drugs from prescribing information (PI) documents, wherein the PI documents comprise information provided by drug regulatory authorities corresponding to the one or more drugs, their uses, and side effects; and
educational information relating to the one or more drugs, wherein the educational information comprises publications relating to the one or more drugs and medical conditions associated with the one or more drugs from a plurality of pre-identified web sources;
an expert input module coupled to the at least one processor to receive expert inputs relating to the information retrieved by the communication module;
an information aggregation module coupled to the at least one processor, wherein the information aggregation module is to, based on the expert inputs, create and store, a drug information database comprising information retrieved by the communication module and processed based on the expert inputs; and
a side effect information module coupled to the at least one processor, the side effect information module is to provide side effects information based on the drug information database, the side effects information being indicative of a side effect of at least one of the one or more drugs.

2. The drug information aggregation system according to claim 1, wherein the communication module is to further interact with at least one of:
mechanism-of-action information relating to the one or more drugs, wherein the mechanism-of-action information comprises mechanism-of-action of at least one molecule present in the one or more drugs;
emerging trends information relating to one or more medical conditions associated with the one or more drugs, wherein the emerging trends information comprises national and international clinical guidelines for the one or more medical conditions; and
drug history information relating to the one or more drugs, wherein the drug history information is indicative of a history of development of the one or more drugs.

3. The drug information aggregation system according to claim 1, further comprising an interaction information module coupled to the at least one processor, wherein the interaction information module is to provide drug interaction information based on the drug information database, the drug interaction information being indicative of a change in a pharmacokinetic parameter of at least one of the one or more drugs when the at least one of the one or more drugs is consumed with at least one additional drug or supplement.

4. The drug information aggregation system according to claim 3, further comprising a user interaction module coupled to the at least one processor, wherein the user interaction module is to:
receive an indication of at least one drug and an additional medication or supplement from a user; and
provide the drug interaction information based on the received indication for consideration of a healthcare provider.

5. The drug information aggregation system according to claim 1, wherein the communication module is to further retrieve image information comprising attributes of image of packaging of various forms and dosages of a plurality of known drugs, the attributes comprising one or more of size, color, marking, volume, and shape of the packaging, wherein the image information is stored in the drug information database.

6. The drug information aggregation system according to claim 1, wherein the information aggregation module comprises:
an artificial intelligence (AI) module, wherein the AI module uses a natural language processing model to construct the drug information database based on the information retrieved by the communication module.

7. The drug information aggregation system according to claim 1, wherein the side effects information further comprises a description of an alternate course of action according to a type of side effect for consideration of the healthcare provider.

8. The drug information aggregation system according to claim 3, wherein the drug interaction information further comprises a description of an alternate course of action according to a type of interaction for consideration of the healthcare provider.

9. The drug information aggregation system according to claim 3, wherein the drug interaction information comprises a graphical representation of potential interaction between the one or more drugs and the at least one additional drug, food, or supplement.

10. The drug information aggregation system according to claim 1, further comprising a patient identification module, wherein the patient identification module is to:
communicate with one or more plan servers of providers of the one or more healthcare plans to identify one or more patients who are registered with at least one of the one or more healthcare plans and are prescribed more than a threshold number of drugs; and
generate an enrollment request for the identified one or more patients to register with the drug information aggregation system,
wherein, upon the enrollment request being accepted by the respective identified patients:
the side effect information module is to provide the side effects information, and
the interaction information module is to provide the drug interaction information.

11. The drug information aggregation system according to claim 10, wherein the patient identification module is further to send prompts to the identified one or more patients upon the enrollment request being accepted, for:
setting up a user password; and
setting up a mode of communication, wherein the mode of communication is selected from a group of email, mail, text, phone, call, and push notifications.

12. The drug information aggregation system according to claim 1, further comprising a drug identification module coupled to the at least one processor to:
receive an image of the one or more drugs to be identified from a user;
extract attributes of the image, wherein the attributes of the image comprise at least one of size, color, marking, volume, and shape; and
identify the one or more drugs based on the attributes using the drug information database to indicate a brand name of the one or more drugs to the user.

13. The drug information aggregation system according to claim 12, wherein the drug identification module is to indicate alternate brand names associated with the one or more drugs.

14. A method for information aggregation, comprising:
accessing health data of a patient based on one or more healthcare plan of the patient, the health data of the patient comprising clinical demographic data, wherein the clinical demographic data comprises information relating to one or more drugs prescribed to the patient,
interacting with a plurality of publicly and privately accessible information sources to retrieve at least one of:
drug approval information relating to the one or more drugs from prescribing information (PI) documents, wherein the PI documents comprise information provided by drug regulatory authorities corresponding to the one or more drugs, their uses, and side effects; and
educational information relating to the one or more drugs, wherein the educational information comprises publications relating to the one or more drugs and medical conditions associated with the one or more drugs from a plurality of preidentified web sources;
receiving expert inputs relating to the information retrieved by interacting with the plurality of publicly and privately accessible information sources;
creating and storing, based on the expert inputs, a drug information database comprising information retrieved from the plurality of publicly and privately accessible information sources and processed based on the expert inputs; and
providing side effects information based on the drug information database, the side effects information being indicative of side effects of at least one of the one or more drugs.

15. The method according to claim 14, further comprising interacting with the plurality of publicly and privately accessible information sources to retrieve at least one of:
mechanism-of-action information relating to the one or more drugs, wherein the mechanism-of-action information comprises mechanism-of-action of at least one molecule present in the one or more drugs;
emerging trends information relating to one or more medical conditions associated with the one or more drugs, wherein the emerging trends information comprises national and international clinical guidelines for the one or more medical conditions; and
drug history information relating to the one or more drugs, wherein the drug history information comprises one or more of: drug class, name, date of approval by the drug regulatory authorities, date of introduction for public use, name of manufacturers, original manufacturer, pricing, and number of clinical publications.

16. The method according to claim 14, further comprising providing drug interaction information based on the drug information database, the drug interaction information being indicative of a change in a pharmacokinetic parameter of at least one of the one or more drugs when the at least one of the one or more drugs is consumed with at least one additional drug or supplement.

17. The method according to claim 16, further comprising:
receiving an indication of at least one drug and an additional medication or supplement from a user; and
providing the drug interaction information based on the received indication for consideration of a healthcare provider.

18. The method according to claim 14, further comprising:
retrieving image information comprising attributes of image of packaging of various forms and dosages of a plurality of known drugs, the attributes comprising one or more of size, color, marking, volume, and shape of the packaging, wherein the image information is stored in the drug information database.

19. The method according to claim 14, further comprising:
communicating with one or more plan servers of providers of the one or more healthcare plans to identify one or more patients who are registered with at least one of the one or more healthcare plans and are prescribed more than a threshold number of drugs; and
generating an enrollment request for the identified one or more patients to register with a system to provide drug aggregation information,
wherein, upon the enrollment request being accepted by the respective identified patients:
providing the side effects information, and
providing the drug interaction information.

20. The method according to claim 19, further comprising sending prompts to the identified patients upon the enrollment request being accepted, for:
setting up a user password; and
setting up a mode of communication, wherein the mode of communication is selected from a group of email, mail, text, phone, call, and a push notification.

21. The method according to claim 14, further comprising performing an identification of one or more drugs, wherein performing the identification of the one or more drugs comprises:
receiving an image of the one or more drugs to be identified from a user;
extracting attributes of the image, wherein the attributes of the image comprise at least one of size, color, marking, volume, and shape; and
identifying the one or more drugs based on the attributes using the drug information database to indicate a brand name of the one or more drugs to the user.

22. A non-transitory computer-readable medium comprising computer-readable instructions executable by a processing resource to:
access health data of patients based on healthcare plans of the patients, the health data of a patient comprising clinical demographic data, wherein the clinical demographic data comprises information relating to one or more drugs prescribed to the patient,
interact with a plurality of publicly and privately accessible information sources to retrieve at least one of:
drug approval information relating to the one or more drugs from prescribing information (PI) documents, wherein the PI documents comprise information provided by drug regulatory authorities corresponding to the one or more drugs, their uses, and side effects; and
educational information relating to the one or more drugs, wherein the educational information comprises publications relating to the one or more drugs and medical conditions associated with the one or more drugs from a plurality of preidentified web sources;
receive expert inputs relating to the information retrieved by interacting with the plurality of publicly and privately accessible information sources;
create and store, based on the expert inputs, a drug information database comprising information retrieved from the plurality of publicly and privately accessible information sources and processed based on the expert inputs; and
provide side effects information based on the drug information database, the side effects information being indicative of side effects of at least one of the one or more drugs.
